(19) United States
(12) Patent Application Publication
(10) Pub. No.: US 2009/0123391 A1
(43) Pub. Date: May 14, 2009

(54) Novel Formulations

(75) Inventors: John T. Capecchi, Oakdale, MN (US); James S. Stefely, Woodbury, MN (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, St. Paul, MN (US)

(21) Appl. No.: 12/293,205

(22) PCT Filed: Mar. 21, 2007
(86) PCT No.: PCT/US2007/064512

§ 371 Date: Sep. 16, 2008

Related U.S. Application Data
(60) Provisional application No. 60/784,670, filed on Mar. 22, 2006.

(51) Int. Cl. A61K 9/12; A61P 11/00

(52) U.S. Cl. 424/45

(57) ABSTRACT

A pharmaceutical aerosol formulation includes a therapeutically effective amount of particulate medicament of formula (I) or a solvate thereof, a propellant selected from the group consisting of 1,1,1,2-tetrafluoroethane, 1,1,1,2,3,3,3-heptafluoro-n-propane or mixtures thereof, and a biocompatible polymer comprising one or more compounds of formula (II)

### CROSS-REFERENCE TO RELATED APPLICATIONS

This application claims priority to U.S. Provisional Patent Application Ser. No. 60/784,670, filed Mar. 22, 2006, the entire contents of which are hereby incorporated by reference.

### FIELD OF THE INVENTION

The present invention relates to novel pharmaceutical aerosol formulations, processes for their preparation, their use in therapy, metered dose inhalers containing said formulations and the use of biocompatible polymers in reducing the variability in the content uniformity and/or in providing enhanced fine particle fraction (FPF) in said formulations.

### BACKGROUND OF THE INVENTION

The delivery of medicinal formulations, comprising for example a drug suspended or dissolved in a carrier, to the lungs by way of inhalation is an important means for treating a variety of conditions, including such common conditions as bronchial asthma and chronic obstructive pulmonary disease. Steroids, β₂-adrenoreceptor agonists, and anti-cholinergic agents are among the drugs that are administered to the lung. Such drugs are commonly administered in aerosol formulations comprising the medicament, one or more propellants and a surfactant and/or a co-solvent, such as ethanol.

WO02/12265 and WO02/12266 disclose novel anti-inflammatory and anti-allergic compounds of the androstane series including a compound of formula (I)

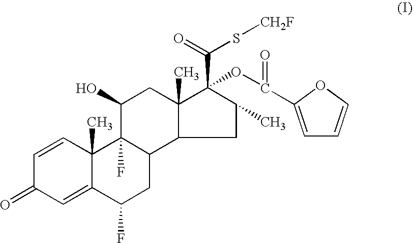

or a solvate thereof, for the treatment and/or prophylaxis of diseases such as asthma and COPD. It is desirable to provide a pharmaceutical aerosol formulation of a compound of formula (I).

Inhaled medicinal aerosol formulations may be formulated as suspensions containing one or more hydrofluoroalkane (HFA) propellants, for example 1,1,1,2-tetrafluoroethane (HFA 134a) and 1,1,1,2,3,3,3-heptafluoro-n-propane (HFA 227).

It is important for commercial purposes that the prescribed dose of aerosol medication delivered from the metered dose inhaler (MDI) to the patient consistently meets the specifications claimed by the manufacturer and complies with the requirements of the FDA and other regulatory authorities. That is, every dose dispensed from the can should be the same within close tolerances. Therefore it is important that the formulation be substantially homogenous throughout the canister and the administered dose at the time of actuation of the metering valve remains similar within close tolerances even after storage. Thus the uniformity of the dose dispensed through the life of the commercially marketed device is important.

The problem of aggregation of the particulate drug may be manifest as a reduction in fine particle fraction (FPF) after storage. The FPF is a measure of the dose dispensed which has the potential to reach the therapeutic portion of the lung. Thus a significant reduction in FPF means that the therapeutically effective amount of drug available to the patient is reduced, which is undesirable and may ultimately be dangerous.

Suspension formulations that are not stabilised adequately often result in high levels of drug deposition. Drug deposition may be on the canister walls or on components of the metered dose inhaler, such as the valve components including the metering chamber or the seals. This deposition may not only result in drug loss thereby reducing the total drug content of the canister available to the patient but also can adversely affect the functioning of the device, resulting in the valve sticking, orifices becoming blocked or caking of drug. Caked drug may work free subsequently so increasing the dose given to the patient in an unpredictable way. Furthermore, extensive modifications to the canister and/or valve may be required to deal with this deposition.

One of the recognized difficulties in the formulation of medicinal suspensions has been the difficulty in dissolving sufficient quantities of surfactants in various hydrofluoroalkane (HFA) propellants, such as HFA 134a and HFA 227. Surfactants generally used with chlorofluorocarbon propellants, for example oleic acid, do not dissolve sufficiently in HFA 134a or HFA 227.

A number of medicinal aerosol formulations using such propellant systems are disclosed in, for example, EP0372777, WO91/04011, WO91/11173, WO91/11495, WO91/14422 and WO92/00061. These applications are concerned with the preparation of pressurised aerosols for the administration of medicaments by inhalation and seek to overcome the problems associated with the use of HFA propellants in the formulations, in particular the problem of instability. The addition of one or more adjuvants such as alcohols, alkanes, dimethyl ether, surfactants (including fluorinated surfactants, carboxylic acids and certain polyethoxylates) and even small amounts of conventional chlorofluorocarbon propellants have been proposed.

There is a need for adjuvants which improve content uniformity and/or FPF of aerosol formulations comprising a compound of formula (I), notwithstanding the teachings of WO98/34596, which relates to the use of relatively low molecular weight biocompatible, preferable biodegradable, polymeric compounds for pharmaceutical drug delivery formulations, or WO94/21229, which discloses medicinal aerosol formulations containing a particulate drug and a dispersing aid derived from a hydroxyacid, a mercapto acid, or an amino acid.

### SUMMARY OF THE INVENTION

The present invention is set forth in an attempt to address the issues in the prior art. In one aspect, the invention provides a pharmaceutical aerosol formulation comprising:

i) a therapeutically effective amount of particulate medicament of formula (I)

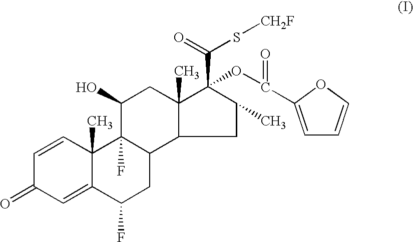

or a solvate thereof;

(ii) a propellant selected from the group consisting of 1,1,1,2-tetrafluoroethane or 1,1,1,2,3,3,3-heptafluoro-n-propane or mixtures thereof; and

(iii) a biocompatible polymer comprising one or more compounds of formula (II)

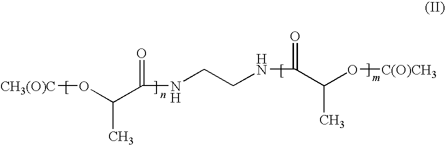

wherein n and m independently represent an integer of at least one and the independent average value of n and m in the biocompatible polymer is between 6 and 25; and each unit of formula

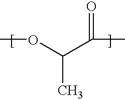

is independently in the D or L configuration.

This aspect and further aspects are contemplated by the present invention and are incorporated herein.

### BRIEF DESCRIPTION OF THE FIGURES

FIG. 1 shows the effect of a biocompatible polymer comprising compounds of formula (II) on mean dose delivered through the valve and %FPF (Anderson Cascade Impactor stages 3-5, approximate aerodynamic diameter 1.1-4.7 μm) for a compound of formula (I), the data was collected using an Anderson Cascade Impactor, at the beginning of use.

FIGS. 2 and 3 show the effect of a biocompatible polymer comprising compounds of formula (II) on mean dose delivered through the valve and %FPF on a combination of a compound of formula (I) and a β₂-adrenoreceptor agonist (Compound B), the data was collected using an Anderson Cascade Impactor, at the beginning of use.

FIGS. 4 and 5 show the effect of a biocompatible polymer comprising compounds of formula (II) on mean dose delivered through the valve and %FPF on a combination of a compound of formula (I) and a β₂-adrenoreceptor agonist (Compound C), the data was collected using an Anderson Cascade Impactor, at the beginning of use.

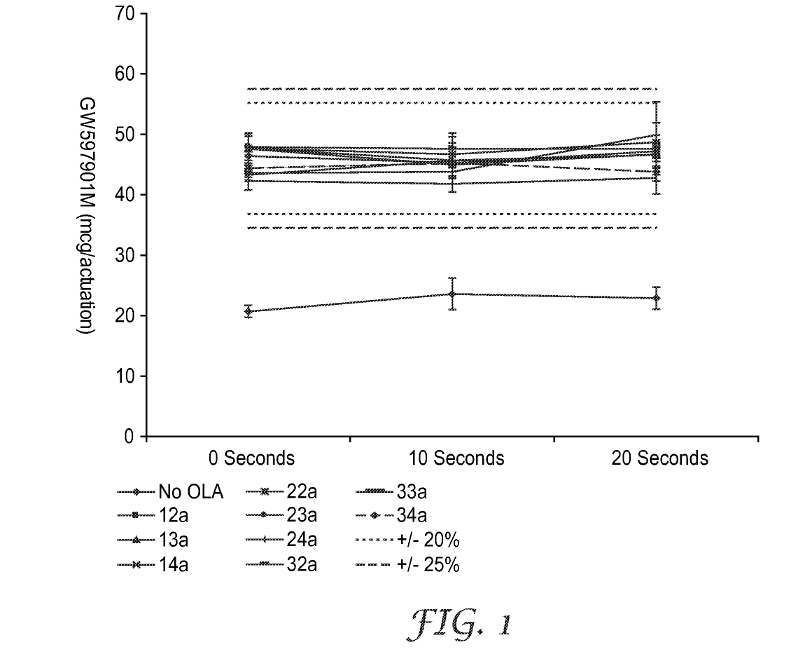

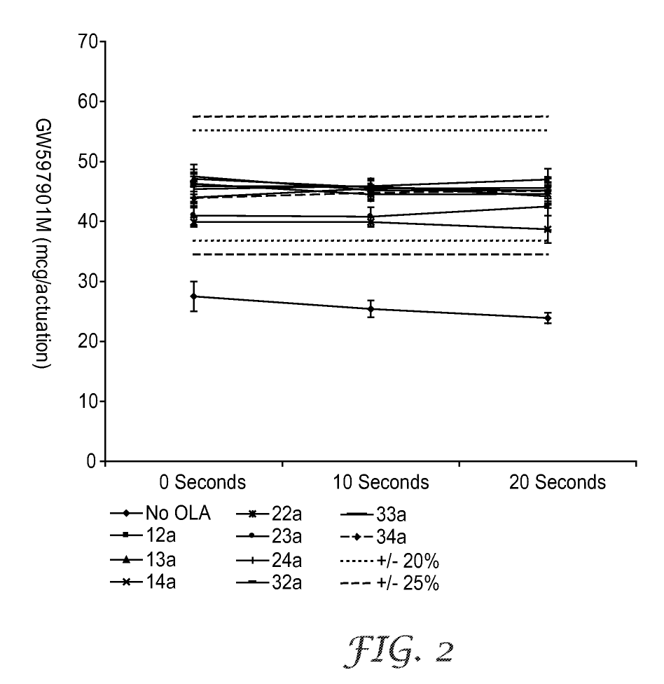

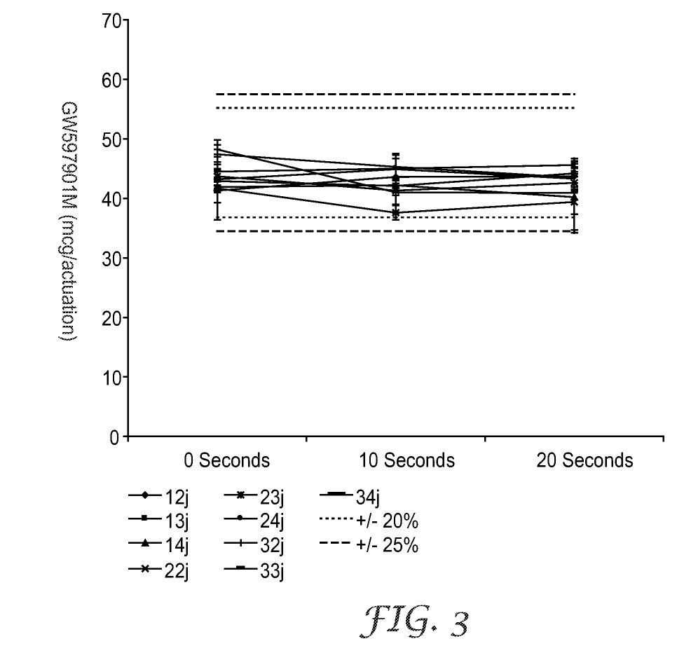

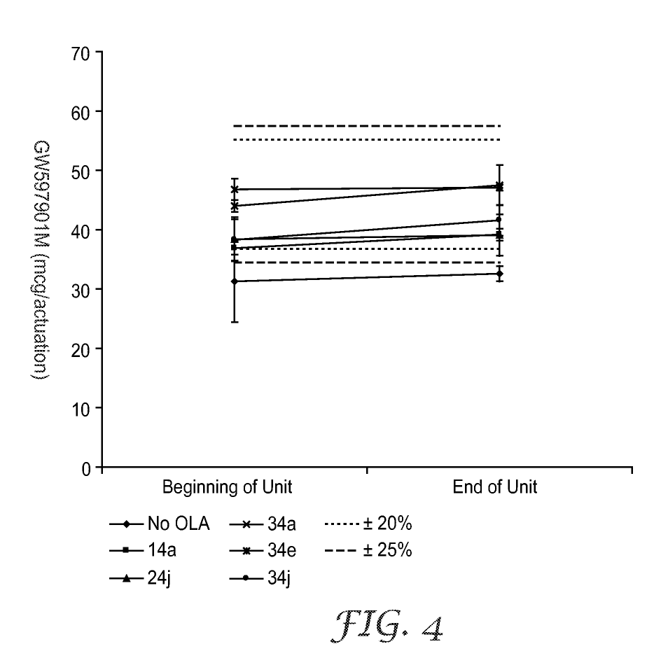

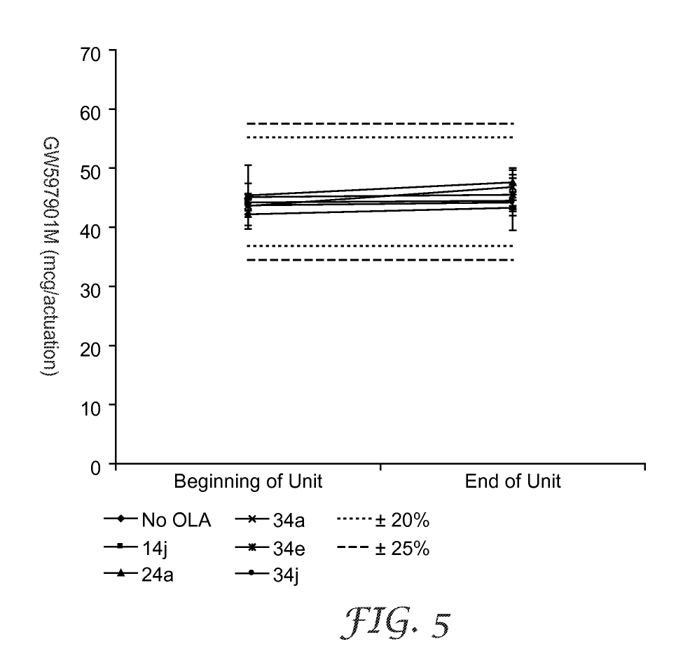

### DETAILED DESCRIPTION OF THE INVENTION

In some embodiments of the invention the independent average value of n and m in the biocompatible polymer is between 7 and 11.

In another aspect of the invention, the pharmaceutical aerosol formulation consists essentially of:

(i) a therapeutically effective amount of particulate medicament of formula (I) or a solvate thereof;

(ii) a propellant selected from the group consisting of 1,1,1,2-tetrafluoroethane, 1,1,1,2,3,3,3-heptafluoro-n-propane or mixtures thereof; and

(iii) a biocompatible polymer comprising one or more compounds of formula (II).

In another aspect of the invention, the pharmaceutical aerosol formulation consists of:

(i) a therapeutically effective amount of particulate medicament of formula (I) or a solvate thereof;

(ii) a propellant selected from the group consisting of 1,1,1,2-tetrafluoroethane, 1,1,1,2,3,3,3-heptafluoro-n-propane or mixtures thereof; and

(iii) a biocompatible polymer comprising one or more compounds of formula (II)

As mentioned above, pharmaceutical aerosol formulations described herein, may be useful in human or veterinary medicine, in particular in the treatment human or animal subjects with inflammatory and/or allergic conditions.

There is thus provided as a further aspect of the invention a pharmaceutical aerosol formulation, as hereinbefore described, for use in human or veterinary medicine, particularly in the treatment of human or animal subjects with inflammatory and/or allergic conditions.

According to another aspect of the invention, there is provided the use of a pharmaceutical aerosol formulation, as hereinbefore described, for the manufacture of a medicament for the administration by inhalation for the treatment of respiratory disorders, for example inflammatory and/or allergic conditions such as asthma or COPD.

In a further aspect, there is provided a method for the treatment and/or prophylaxis of a respiratory disorder which comprises administering to a human or animal subject a pharmaceutical aerosol formulation, as hereinbefore described.

The pharmaceutical formulation according to the invention may additionally contain one or more other therapeutically active agents, for example selected from other anti-inflammatory agents, anticholinergic agents (particularly an M₁, M₂, M₁/M₂ or M₃ receptor antagonist), β₂-adrenoreceptor agonists, antiinfective agents (e.g. antibiotics, antivirals), or antihistamines.

The invention thus provides, in a further aspect, a pharmaceutical aerosol formulation as hereinbefore described, together with one or more other therapeutically active agents, for example, selected from another anti-inflammatory agent (for example a corticosteroid or an NSAID), an anticholinergic agent, a β₂-adrenoreceptor agonist, an antiinfective agent (e.g. an antibiotic or an antiviral), or an antihistamine. Preferred formulations comprise a compound of formula (I) or a pharmaceutically acceptable salt, solvate or physiologically functional derivative thereof together with a β₂-adrenoreceptor agonist, and/or an anticholinergic, and/or a PDE-4 inhibitor. Preferred combinations are those comprising one or two other therapeutic agents.

It will be clear to a person skilled in the art that, where appropriate, the other therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

A pharmaceutical aerosol formulation comprising a compound of formula (I), as herein before described, together with a β₂-adrenoreceptor agonist is particularly preferred.

Examples of β₂-adrenoreceptor agonists include salmeterol (e.g. as racemate or a single enantiomer such as the R-enantiomer or the S-enantiomer), salbutamol (e.g. as racemate or a single enantiomer such as the R-enantiomer), formoterol (e.g. as racemate or a single enantiomer such as the R,R-enantiomer), fenoterol, carmoterol, etanterol, naminterol, clenbuterol, pirbuterol, flerbuterol, reproterol, bambuterol, terbutaline salmefamol, indacaterol and salts thereof, for example the xinafoate (1-hydroxy-2-naphthalenecarboxylate) salt of salmeterol, the sulphate salt of salbutamol or the fumarate salt of formoterol. Long-acting β₂-adrenoreceptor agonists, for example, compounds which provide effective bronchodilation for about 12 hours or longer, are preferred.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and W003/042160.

Particular β₂-adrenoreceptor agonists include:

3-(4-{[6-({(2R)-2-hydroxy-2-[4-hydroxy-3-(hydroxymethyl)phenyl]ethyl}amino) hexyl] oxy} butyl) benzenesulfonamide;

3-(3-{[7-({(2R)-2-hydroxy-2-[4-hydroxy-3-hydroxymethyl) phenyl] ethyl}-amino) heptyl] oxy} propyl) benzenesulfonamide;

4-{(1R)-2-[(6-{2-[(2, 6-dichlorobenzyl) oxy] ethoxy} hexyl) amino]-1-hydroxyethyl}-2-(hydroxymethyl) phenol;

4-{(1R)-2-[(6-{4-[3-(cyclopentylsulfonyl)phenyl]butoxy}hexyl)amino]-1-hydroxyethyl}-2-(hydroxymethyl)phenol;

N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;

N-{2-[4-(3-phenyl-4-methoxyphenyl)aminophenyl]ethyl}-2-hydroxy-2-(8-hydroxy-2(1H)-quinolinon-5-yl)ethylamine; and

5-[(R)-2-(2-{4-[4-(2-amino-2-methyl-propoxy)-phenylamino]-phenyl}-ethylamino)-1-hydroxy-ethyl]-8-hydroxy-1H-quinolin-2-one;

and pharmaceutically acceptable salts thereof.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Suitable corticosteroids which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester, 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (eg. the 17-propionate ester or the 17,21-dipropionate ester), budesonide, flunisolide, mometasone esters (eg. the furoate ester), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-11β-hydroxy-16α-methyl-3-oxo-17α-(2,2,3,3- tetramethycyclopropylcarbonyl)oxy-androsta-1,4-diene-17β-carbothioic acid S-cyanomethyl ester and 6α,9αa-difluoro-11β-hydroxy-16α-methyl-17α-(1-methycyclopropylcarbonyl )oxy-3-oxo-androsta-1,4-diene-17β-carbothioic acid S-fluoromethyl ester.

Non-steroidal compounds having glucocorticoid agonism that may possess selectivity for transrepression over transactivation and that may be useful in combination therapy include those covered in the following patents: WO03/082827, WO01/10143, WO98/54159, WO04/005229, WO04/009016, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651 and WO03/08277.

Suitable anti-inflammatory agents include non-steroidal anti-inflammatory drugs (NSAID's).

Suitable NSAID's include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis (eg. montelukast), iNOS inhibitors, tryptase and elastase inhibitors, beta-2 integrin antagonists and adenosine receptor agonists or antagonists (e.g. adenosine 2a agonists), cytokine antagonists (e.g. chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Suitable iNOS inhibitors include those disclosed in WO93/13055, WO98/30537, WO02/50021, WO95/34534 and WO99/62875. Suitable CCR3 inhibitors include those disclosed in WO02/26722.

Of particular interest is use of the compounds of formula (I) in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds of interest include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep. 1996; this patent and the compounds it discloses are incorporated herein in full by reference.

Other compounds of interest include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther,1998, 284(1): 162), and T2585.

Further compounds of interest are disclosed in the published international patent application WO04/024728 (Glaxo Group Ltd), PCT/EP2003/014867 (Glaxo Group Ltd) and PCT/EP2004/005494 (Glaxo Group Ltd).

Suitable anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (e.g. as the bromide, CAS 22254-24-6, sold under the name Atrovent), oxitropium (e.g. as the bromide, CAS 30286-75-0) and tiotropium (e.g. as the bromide, CAS 136310-93-5, sold under the name Spiriva). Also of interest are revatropate (e.g. as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (e.g. as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Other suitable anticholinergic agents include compounds of formula (XXI), which are disclosed in U.S. patent application Ser. No. 60/487981:

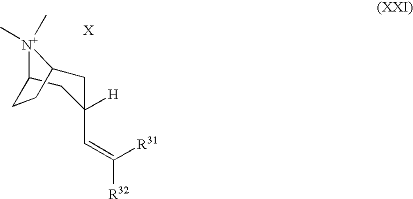

in which the preferred orientation of the alkyl chain attached to the tropane ring is endo; R³¹ and R³² are, independently, selected from the group consisting of straight or branched chain lower alkyl groups having preferably from 1 to 6 carbon atoms, cycloalkyl groups having from 5 to 6 carbon atoms, cycloalkyl-alkyl having 6 to 10 carbon atoms, 2-thienyl, 2-pyridyl, phenyl, phenyl substituted with an alkyl group having not in excess of 4 carbon atoms and phenyl substituted with an alkoxy group having not in excess of 4 carbon atoms;

X⁻ represents an anion associated with the positive charge of the N atom. X⁻ may be but is not limited to chloride, bromide, iodide, sulfate, benzene sulfonate, and toluene sulfonate,

including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further suitable anticholinergic agents include compounds of formula (XXII) or (XXIII), which are disclosed in U.S. patent application Ser. No. 60/511009:

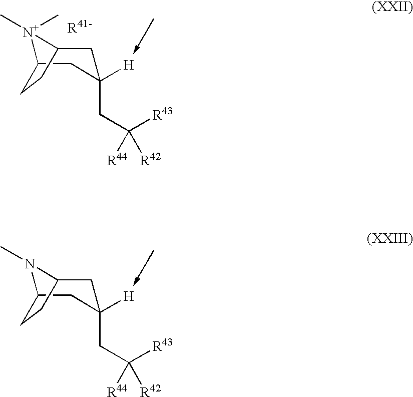

wherein:

the H atom indicated is in the exo position;

R⁴¹ represents an anion associated with the positive charge of the N atom. R⁴¹ may be but is not limited to chloride, bromide, iodide, sulfate, benzene sulfonate and toluene sulfonate;

R⁴² and R⁴³ are independently selected from the group consisting of straight or branched chain lower alkyl groups (having preferably from 1 to 6 carbon atoms), cycloalkyl groups (having from 5 to 6 carbon atoms), cycloalkyl-alkyl (having 6 to 10 carbon atoms), heterocycloalkyl (having 5 to 6 carbon atoms) and N or O as the heteroatom, heterocycloalkyl-alkyl (having 6 to 10 carbon atoms) and N or O as the heteroatom, aryl, optionally substituted aryl, heteroaryl, and optionally substituted heteroaryl;

R⁴⁴ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl, —OR⁴⁵, —CH₂OR⁴⁵, —CH₂OH, —CN, —CF₃, —CH₂O(CO)R⁴⁶, —CO₂R⁴⁷, —CH₂NH₂, —CH₂N(R⁴⁷)SO₂R⁴⁵, —SO₂N(R⁴⁷)(R⁴⁸), —CON(R⁴⁷)(R⁴⁸), —CH₂N(R⁴⁸)CO(R⁴⁶), —CH₂N(R⁴⁸)SO₂(R⁴⁶), —CH₂N(R⁴⁸)CO₂(R⁴⁵), —CH₂N(R⁴⁸)CONH(R⁴⁷);

R⁴⁵ is selected from the group consisting of (C₁-C₆)alkyl, (C₁-C₆)alkyl(C₃-C₁₂)cycloalkyl, (C₁-C₆)alkyl(C₃-C₇)heterocycloalkyl, (C₁-C₆)alkyl-aryl, (C₁-C₆)alkyl-heteroaryl;

R⁴⁶ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl;

R⁴⁷ and R⁴⁸ are, independently, selected from the group consisting of H, (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl (C₃-C₇)heterocycloalkyl, (C₁-C₆)alkyl-aryl, and (C₁-C₆)alkyl-heteroaryl, including, for example:
(Endo)-3-(2-methoxy-2,2-di-thiophen-2-yl-ethyl)-8,8-dimethyl-8-azonia-bicyclo[3.2.1]octane iodide;
3-((Endo)-8-methyl-8-aza-bicyclo[3.2.1]oct-3-yl)-2,2-diphenyl-propionitrile;
(Endo)-8-methyl-3-(2,2,2-triphenyl-ethyl)-8-aza-bicyclo[3.2.1]octane;
3-((Endo)-8-methyl-8-aza-bicyclo[3.2.1]oct-3-yl)-2,2-diphenyl-propionamide;
3-((Endo)-8-methyl-8-aza-bicyclo[3.2.1]oct-3-yl)-2,2-diphenyl-propionic acid;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
3-((Endo)-8-methyl-8-aza-bicyclo[3.2.1]oct-3-yl)-2,2-diphenyl-propan-1-ol;
N-Benzyl-3-((endo)-8-methyl-8-aza-bicyclo[3.2.1]oct-3-yl)-2,2-diphenyl-propionamide;
(Endo)-3-(2-carbamoyl-2,2-diphenyl-ethyl)-8,8-dimethyl-8-azonia-bicyclo[3.2.1]octane iodide;
1-Benzyl-3-[3-((endo)-8-methyl-8-aza-bicyclo[3.2.1]oct-3-yl)-2,2-diphenyl-propyl]-urea;
1-Ethyl-3-[3-((endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-acetamide;
N-[3-((Endo)-8-methyl-8-aza-bicyclo[3.2.1]oct-3-yl)-2,2-diphenyl-propyl]-benzamide;
3-((Endo)-8-methyl-8-aza-bicyclo[3.2.1]oct-3-yl)-2,2-di-thiophen-2-yl-propionitrile;
(Endo)-3-(2-cyano-2,2-di-thiophen-2-yl-ethyl)-8,8-dimethyl-8-azonia-bicyclo[3.2.1]octane iodide;
N-[3-((Endo)-8-methyl-8-aza-bicyclo[3.2.1]oct-3-yl)-2,2-diphenyl-propyl]-benzenesulfonamide;
[3-((Endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-methanesulfonamide; and/or
(Endo)-3-{2,2-diphenyl-3-[(1-phenyl-methanoyl )-amino]-propyl}-8,8-dimethyl-8-azonia-bicyclo[3.2.1]octane bromide.

More preferred compounds useful in the present invention include:
(Endo)-3-(2-methoxy-2,2-di-thiophen-2-yl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
(Endo)-3-(2-carbamoyl-2,2-diphenyl-ethyl)-8,8-dimethyl-8-azonia-bicyclo[3.2.1]octane iodide;
(Endo)-3-(2-cyano-2,2-di-thiophen-2-yl-ethyl)-8,8-dimethyl-8-azonia-bicyclo[3.2.1]octane iodide; and/or
(Endo)-3-{2,2-diphenyl-3-[(1-phenyl-methanoyl)-amino]-propyl}-8,8-dimethyl-8-azonia-bicyclo[3.2.1]octane bromide.

Suitable antihistamines (also referred to as H₁-receptor antagonists) include any one or more of the numerous antagonists known which inhibit H₁-receptors, and are safe for human use. First generation antagonists, include derivatives of ethanolamines, ethylenediamines, and alkylamines, e.g diphenylhydramine, pyrilamine, clemastine, chloropheniramine. Second generation antagonists, which are non-sedating, include loratidine, desloratidine, terfenadine, astemizole, acrivastine, azelastine, levocetirizine fexofenadine and cetirizine.

Examples of preferred anti-histamines include loratidine, desloratidine, fexofenadine and cetirizine.

In formulations of the present invention the biocompatible polymer comprising one or more compounds of formula (II) is considered to have good surfactant properties. These surfactant properties may include reducing the deposition on the internal surfaces of the can thereby increasing the amount of drug that comes through the valve, stabilising, enhancing and reducing variability in the fine particle fraction (FPF), giving good content uniformity performance by reducing variability in delivered dose uniformity and reducing the product overage required to achieve the delivered dose. The biocompatible polymer comprising one or more compounds of formula (II) in the formulations of the present invention is considered to be advantageous in terms of improving the stability of the aerosol formulation by reducing drug deposition, increasing shelf life and the like.

In one aspect of the invention there is provided a pharmaceutical aerosol formulation wherein the particulate medicament of formula (I) is 6α, 9α-difluoro-17α-[(2-furanylcarbonyl )oxy]-11β-hydroxy-16α-methyl-3-oxo-androsta-1,4-diene-17β-carbothioic acid S-fluoromethyl ester.

In some embodiments of the invention is provided a pharmaceutical aerosol formulation wherein the particulate medicament of formula (I) is in unsolvated form.

In some embodiments of the invention is provided a pharmaceutical aerosol formulation wherein the particulate medicament of formula (I) is in the form of Form 1 polymorph.

WO02/12265 and WO02/12266 disclose compounds of formula (I), including solvates, unsolvated Forms and Form 1 polymorphs, these applications are incorporated herein by reference.

In one aspect of the invention there is provided a pharmaceutical aerosol formulation as described hereinbefore further comprising 3-(4-{[6-({(2R)-2-hydroxy-2-[4-hydroxy-3-(hydroxymethyl)phenyl]ethyl}amino)hexyl] oxy} butyl) benzenesulfonamide.

In another aspect of the invention there is provided a pharmaceutical aerosol formulation as described hereinbefore further comprising 3-(3-{[7-({(2R)-2-hydroxy-2-[4-hydroxy-3-hydroxymethyl) phenyl] ethyl}-amino) heptyl] oxy} propyl) benzenesulfonamide.

In another aspect of the invention there is provided a pharmaceutical aerosol formulation as described hereinbefore further comprising

4-{(1R)-2-[(6-{2-[(2, 6-dichlorobenzyl) oxy] ethoxy} hexyl) amino]-1-hydroxyethyl}-2-(hydroxymethyl) phenol.

In another aspect of the invention there is provided a pharmaceutical aerosol formulation as described hereinbefore further comprising

4-{(1R)-2-[(6-{4-[3-(cyclopentylsulfonyl)phenyl]butoxy}hexyl)amino]-1-hydroxyethyl}-2-(hydroxymethyl)phenol.

In another aspect of the invention there is provided a pharmaceutical aerosol formulation as described hereinbefore further comprising N-[2-hydroxyl-5-[(1R)-1-hydroxy-2-[[2-4-[[(2R)-2-hydroxy-2-phenylethyl]amino]phenyl]ethyl]amino]ethyl]phenyl]formamide.

In another aspect of the invention there is provided a pharmaceutical aerosol formulation as described hereinbefore further comprising N-{2-[4-(3-phenyl-4-methoxyphenyl)aminophenyl]ethyl}-2-hydroxy-2-(8-hydroxy-2(1H)-quinolinon-5-yl)ethylamine.

In further aspect of the invention there is provided a pharmaceutical aerosol formulation as described hereinbefore further comprising 5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The biocompatible polymer comprising one or more compounds of formula (II) may be prepared by a number of reaction methods, such as those disclosed in WO94/21229 and WO98/34596. In one embodiment, lactic acid may be polymerised via condensation followed by capping the hydroxyl end of the polymer with an acetyl capping group. Ethylenediamine can then be coupled to the oligolactic acid via condensation and formation of an amide.

These reactions may be run in solution, and the solvent may also serve as the propellant in the formulation, if applicable. Preferred solvents that may also serve as propellants include HFA 134a and HFA 227. Examples of suitable synthetic methods for polymerizing and capping polymers may be found in U.S. patent application Ser. Nos. 60/533172 (“Medicinal Compositions and Method for the Preparation Thereof”, Capecchi et al.) and 60/613063 (“Medicinal Aerosol Formulations and Methods of Synthesizing Ingredients Therefor”, Bechtold et al.), the disclosures of which are herein incorporated by reference.

The method of polymer condensation, as described in U.S. patent application Ser. No. 60/533172, is considered to provide significant advantages. Besides the unexpected superiority of the products, it is also considered to provide advantages over other polymerizations that utilize metal-based catalysts, which are more expensive, present environmental disadvantages, and raise health concerns due to residual contamination. It may also provide improved degrees of acylation or acetylation of the OH endgroups and of the degree of derivatization of the acid functionality with a capping or bridging group, such as ethylenediamine. In one aspect, the reaction method provides for degrees of completion such that the molar ratio of unreacted oligolactic acid and oligolactic acid derivatives having a free hydroxyl is less than 10%, less than 5%, or less than 1% of the amount of N,N′-ethylenebis (acetyloligolactyl) amide prepared. In one aspect, the reaction method also provides for degrees of completion such that the molar ratio of unreacted oligolactic acid and oligolactic acid derivatives having a free carboxylic acid is also less than 10%, less than 5%, or less than 1% of the amount of N,N′-ethylenebis (acetyloligolactyl) amide prepared. Determination of the relative amount of unreacted oligolactic acid and oligolactic acid derivatives having a free carboxylic acid may be determined by conventional analytical methods, such as, for example, nuclear magnetic resonance (NMR) or liquid chromatography-mass spectrometry (LC-MS).

Use of said biocompatible polymer for the preparation of a formulation according to the present invention is believed to result in effective suspension stabilisation and reduction in drug deposition. Thus, the amount of biocompatible polymer employed is desirably in the range of from 0.0025% to 3% w/w, particularly from 0.01% to 0.5% w/w, more particularly from 0.05% to 0.2% w/w, relative to the propellant.

The particle size of the particulate (e.g. micronised) medicament should be such as to optimise the amount of the medicament inhaled into the lungs upon administration of the aerosol formulation and will thus be less than 100 microns, desirably less than 20 microns, and preferably will have a MMAD (mass median aerodynamic diameter) in the range 1-10 microns, e.g. 1-5 microns.

The final aerosol formulation desirably contains 0.005-10% w/w, preferably 0.005-5% w/w, especially 0.01-1.0% w/w, of medicament relative to the total weight of the formulation.

Administration of medicament may be indicated for the treatment of mild, moderate or severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1, 2, 3 or 4 puffs each time.

Suitable daily doses, may be, for example in the range 25 to 800 microgram for a compound of formula (I), 5 to 20 microgram for Compound B, 10 to 50 microgram for Compound C, depending on the severity of the disease.

Typically each filled canister for use in a metered dose inhaler contains 100, 160 or 240 metered doses or puffs of medicament.

In some embodiments a single propellant is employed, for example, 1,1,1,2-tetrafluoroethane or 1,1,1,2,3,3,3-heptafluoro-n-propane, suitably 1,1,1,2-tetrafluoroethane.

It is desirable that the formulations of the invention contain no components which may provoke the degradation of stratospheric ozone. In particular it is desirable that the formulations are substantially free of chlorofluorocarbons such as CCl₃F, CCl₂F₂ and CF₃CCl₃.

If desired the propellant may additionally contain a volatile adjuvant such as a saturated hydrocarbon, for example, propane, n-butane, isobutane, pentane and isopentane or a dialkyl ether, for example, dimethyl ether. In general, up to 50% w/w of the propellant may comprise a volatile hydrocarbon, for example, 1 to 30% w/w. However, formulations which are substantially free of volatile adjuvants may be preferred. In certain cases, it may be desirable to include appropriate amounts of water, which can be advantageous in modifying the dielectric properties of the propellant.

Polar adjuvants which may, if desired, be incorporated into the formulation according to the present invention include, for example, C₂₋₆aliphatic alcohols and polyols such as ethanol, isopropanol and propylene glycol and mixtures thereof. Typically ethanol will be employed. In general only small quantities (e.g. from 0.05 to 3.0% w/w) of polar adjuvants are required and the use of quantities in excess of 5% w/w may disadvantageously tend to dissolve the medicament. Formulations preferably contain less than 1% w/w, for example, about 0.1% w/w of polar adjuvant. Most preferably the formulations according to the invention are substantially free of polar adjuvant. Polarity may be determined, for example, by the method described in European Patent Application Publication No. 0327777.

In various optional embodiments, the formulations may be substantially free of: (1) volatile adjuvants, for example, saturated hydrocarbons such as, without limitation, propane, n-butane, isobutane, pentane, isopentane or a dialkyl ether, for example, dimethyl ether, (2) conventional surfactants for example, oleic acid, lecithin and sorbitan trioleate), and/or (3) components of higher polarity, for example, alcohols such as ethanol. For the purposes of the invention, the term “substantially free” refers to the above component(s) being present in an amount below detectable limit.

The formulation according to the present invention may optionally contain one or more further ingredients conventionally used in the art of pharmaceutical aerosol formulation. Such optional ingredients include, but are not limited to, taste masking agents, sugars, buffers, antioxidants, water and chemical stabilisers.

The invention also extends to formulations as described already which consist rather than comprise said elements.

A further embodiment of the invention is a sealed container capable of withstanding the pressure required to maintain the propellant as a liquid, such as a metered dose inhaler, containing therein the aerosol formulation as described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI cans generally comprise a container capable of withstanding the vapour pressure of the propellant used such as a plastic or plastic-coated glass bottle or preferably a metal can, for example, stainless steel, aluminium or an alloy thereof which may optionally be anodised, lacquer-coated and/or plastic-coated (e.g. incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g., see Byron, above and WO96/32099). Preferably the canister is fitted with a cap assembly, wherein a drug metering valve is situated in the cap, and said cap is crimped in place.

In one embodiment of the invention the metallic internal surface of the can is coated with a fluoropolymer, most preferably blended with a non-fluoropolymer. In another embodiment the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

Formulations according to the present invention may obviate the need for the additional processing of the canisters and/or component by coating, for example, which may lead to cost saving, especially when manufacturing in bulk.

The metering valves are designed to deliver a metered amount of the formulation per actuation and may incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; 6,679,374, as well as dose counter units such as, but not limited to, those described in U.S. Pat. Nos. 6,360,739 and 6,431,168.

The formulations of the invention may be prepared by dispersal of the medicament of formula (I) and the biocompatible polymer of formula (II) in the propellant in an appropriate container, for example, with the aid of sonication or a high-shear mixer. This process is desirably carried out under controlled humidity conditions.

A further aspect of this invention comprises a process for filling the said formulation into MDIs.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The particulate medicament is added to a charge vessel and liquefied propellant is pressure filled through the charge vessel into a manufacturing vessel, together with liquefied propellant containing the surfactant. The drug suspension is mixed before recirculation to a filling machine and an aliquot of the drug suspension is then filled through the metering valve into the canister.

In an alternative process, an aliquot of the liquefied formulation is added to an open canister under conditions which are sufficiently cold to ensure formulation does not vaporise, and then a metering valve crimped onto the canister.

Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler system for administration of the medicament into the lungs or nasal cavity of a patient.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The fine particle fraction of the aerosol formulations according to the invention may be measured by conventional techniques, for example, by cascade impaction by measuring particle size distribution. The Cascade Impactor is designed to mimic the human buccal cavity and bronchial tract and the cascade Impactor test is designed to reveal the amount of deposition of inhaled drug substance at various stages thereof. As used herein reference to the “cascade impaction” assay means determination of the deposition of the emitted dose in pressurised inhalations as defined in European Pharmacopoeia, Section 2.9.18, 5^{th} edition “Preparations for Inhalation, Apparatus D”. Such techniques enable the “respirable fraction” of the aerosol formulations to be calculated. One method used to calculate the “respirable fraction” is by reference to “fine particle fraction” which is the amount of active ingredient collected in stages 3 to 5 (aerodynamic diameter 1.1-4.7 μm) which represents the lung, per actuation expressed as a percentage of the total amount of active ingredient delivered per actuation using the cascade impactor method described above. Earlier stages represent the aerosol device itself, the throat and the upper reaches of the bronchial tract, and the later stages representing potential systemic absorption through the wall of the lung which may cause serious side effects.

Metered dose inhalers are designed to deliver a fixed unit dosage of medicament per actuation or “puff”, for example, in the range of 10 to 5000 micrograms of medicament per puff.

Administration of medicament may be indicated for the treatment of mild, moderate, severe acute or chronic symptoms or for prophylactic treatment. It will be appreciated that the precise dose administered will depend on the age and condition of the patient, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example, from 1 to 8 times per day, giving for example 1, 2, 3 or 4 puffs each time.

An appropriate dosing regime for other medicaments will be known or readily available to persons skilled in the art.

Another aspect of the invention involves the use of biocompatible polymer of formula (II) to enhance the FPF or reduce the variability in the content uniformity, for example, by reducing the relative standard deviation (RDS) of the individual emitted dose.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The following non-limiting examples serve to illustrate the invention.

### EXAMPLES

### Dose Through Unit (DTU) Method Procedures

The dose collection apparatus (500 ml separatory funnel with a cotton plug) was assembled and the flow rate was set to 20 L/min. Test units were stored at ambient conditions for two weeks after manufacture prior to DTU testing. For the testing at the beginning of the unit, the MDIs were primed two times with a priming actuator and four times to waste with the test actuator, shaking the units between each actuation. Two test actuations were collected in the dose collection apparatus, shaking the unit between actuations. The collection apparatus was rinsed with an appropriate volume of diluent, and the rinsate with the collected dose was analyzed by conventional HPLC analysis. For end of unit testing, the MDIs were actuated an additional 48 times to waste, shaking between each actuation. The MDIs were then actuated four times to waste through a new test actuator. Two test actuations were then collected in the dose collection apparatus, shaking the unit between actuations. The collection apparatus was rinsed with an appropriate volume of diluent, and the rinsate with the collected dose was analyzed by conventional HPLC analysis.

The reported results are the average of ten units at both the beginning and end of unit use.

### Andersen Cascade Impactor (ACI) Method Procedures

The Andersen Cascade Impactor Mark II (ACI) was assembled and the flow rate was set to 28.3 L/min. The units were primed four times with the test actuator prior to dose collection, shaking between actuations. Between 5 and 20 actuations were collected in the ACI assembly. The ACI was disassembled and the components were rinsed with an appropriate amount of solvent that ensures dissolution of all formulation ingredients. The rinsate was collected for analysis by conventional HPLC analysis.

### Test Compounds

The test compounds were as follows:

Compound A—6α, 9α-Difluoro-17α-[(2-furanylcarbonyl)oxy]-11β-hydroxy-16α-methyl-3-oxo-androsta-1,4-diene-17β-carbothioic acid S-fluoromethyl ester.
Compound B—N-[2-hydroxy-5-[(1R)-1-hydroxy-2-[[2-4-[[(2R)-2-hydroxy-2-phenylethyl]amino]phenyl]ethyl]amino]ethyl]phenyl]formamide.
Compound C—3-(4-{[6-({(2R)-2-hydroxy-2-[4-hydroxy-3-(hydroxymethyl)-phenyl]ethyl}amino)hexyl]oxy}butyl)benzenesulfonamide.
Compound D—4-{(1R)-2-[(6-{2-[(2,6-dichlorobenzyl) oxy] ethoxy} hexyl) amino]-1-hydroxyethyl}-2-(hydroxymethyl) phenol.
Compound E—N-{2-[4-(3-phenyl-4-methoxyphenyl)aminophenyl]ethyl}-2-hydroxy-2-(8-hydroxy-2(1H)-quinolinon-5-yl)ethylamine;

### Example 1

Compound A, MDI, 25 μg/act, 60 Actuations

The cold-filling equipment, which comprises a stainless steel batching vessel with an air-driven mixer and filling valve, was assembled. The propellant was chilled to about −60° C. The batching vessel was chilled to at least −30° C. and about half of the total chilled propellant was added. The propellant was allowed to reach at least −50° C. With the mixer running, 1.3013 g of the biocompatible polymer comprising compounds of formula (II) was added for a concentration of 0.1% w/w relative to propellant followed by addition of 0.4294 g of 6α,9α-difluoro-17α-[(2-furanylcarbonyl)oxy]-11β-hydroxy-16α-methyl-3-oxo-androsta-1,4-diene-17β-carbothioic acid S-fluoromethyl ester powder. The remaining cold propellant was then added up to the total weight of 1299 g of HFA 134a, and the containers rinsed to ensure all the powders were added. The suspension was mixed at about 3000 rpm for about 15 minutes. Before filling the MDI units, the formulation temperature was confirmed to be about −60° C. The filling valve was adjusted to deliver the appropriate fill weight. Fluoropolymer coated aluminum canisters were filled to the specific fill weight target, 7.3 g of HFA 1 34a. A Valois DF60 Mark 66 valve was immediately placed on the canister and crimped. Each unit was formulated to deliver a total of about 100 actuations. The formulations were then allowed to warm to room temperature and spray tested two times to waste, in order to ensure that the unit was working correctly.

Comparative surfactant-free formulations were prepared as generally described above with the exception that no biocompatible polymer of formula (II) was added to the formulation.

Dose Uniformity—Compound A, MDI, HFA 134a, 25 μg/act, 60 Actuations

Table 1 shows the overall mean dose of medicament delivered through the actuator, combining beginning and end of use doses. The target output of drug is 22.5 μg/act (assuming 10% actuator deposition).

TABLE 1

| | Surfactant- | 0.1% w/w of polymer |
|---|---|---|
| | free | of formula (II) |
| Initial | 66% | 98% |
| 12 Week at 40° C./75% | 72% | 98% |
| relative humidity | | |

Table 2 shows the variability of individual doses, combining beginning and end of use doses (% Relative Standard Deviation, n=20).

TABLE 2

| | Surfactant- | 0.1% w/w of polymer |
|---|---|---|
| | free | of formula (II) |
| Initial | 7.5% | 4.4% |
| 12 Week at 40° C./75% | 8.1% | 4.3% |
| relative humidity | | |

Fine Particle Fraction—Compound A, MDI, HFA 134a, 25 μg/act, 60 Actuations

Table 3 shows the Fine Particle Fraction (FPF) expressed as percentage of 25 μg, the total dose target.

TABLE 3

| | Surfactant- | 0.1% w/w of polymer |
|---|---|---|
| | free | of formula (II) |
| Initial | 17% | 24% |
| 12 Week at 40° C./75% | 16% | 22% |
| relative humidity | | |

Additional formulations of varying concentrations of biocompatible polymer of the present invention were prepared by similar methods.

### Example 2

Compound A in Combination with Compound B, MDI, 25/10 μg/act, 60 Actuations

The cold-filling equipment, which comprises a stainless steel batching vessel with an air-driven mixer and filling valve, was assembled. The propellant was chilled to about −60° C.

The batching vessel was chilled to at least −30° C. and about half of the total chilled propellant was added. The propellant was allowed to reach at least −50° C. With the mixer running, 1.0379 g of the biocompatible polymer comprising compounds of formula (II) was added for a concentration of 0.1% w/w relative to propellant, followed by the addition of 0.3564 g of 6α,9α-difluoro-17α-[(2-furanylcarbonyl)oxy]-11β-hydroxy-16α-methyl-3-oxo-androsta-1,4-diene-17β-carbothioic acid S-fluoromethyl ester powder and 0.1539 g of N-[2-hydroxy-5-[(1R)-1-hydroxy-2-[[2-4-[[(2R)-2-hydroxy-2-phenylethyl]amino]phenyl]ethyl]amino]ethyl]phenyl]formamide powder. The remaining cold propellant was then added up to the total weight, 1033 g of HFA 134a, and the containers rinsed to ensure all the powders were added. The suspension was mixed at about 3000 rpm for about 15 minutes. Before filling the MDI units, the formulation temperature was confirmed to be about −60° C. The filling valve was adjusted to deliver the appropriate fill weight. Fluoropolymer coated aluminum canisters were filled to the specific fill weight target, 7.3 g of HFA 134a. A Valois DF60 Mark 66 valve was immediately placed on the canister and crimped. Each unit was formulated to deliver a total of about 100 actuations. The formulations were then allowed to warm to room temperature and spray tested two times to waste, in order to ensure that the unit was working correctly.

Comparative surfactant-free formulations were prepared as generally described above with the exception that no biocompatible polymer of formula (II) was added to the formulation.

Dose Uniformity—Compound A in Combination with Compound B, MDI, HFA 134a, 25/10 μg/act, 60 Actuations

Table 4 shows the overall mean dose of medicament delivered through the actuator, combining beginning and end of use doses.

The target output of Compound B is 8.5 μg (assuming 15% actuator deposition).

The target output of Compound A is 22.5 μg (assuming 10% actuator deposition).

TABLE 4

| | Compound B Component | | Compound A Component | |
|---|---|---|---|---|
| | | 0.1% w/w of | | 0.1% w/w of |
| | Surfactant- | polymer of | Surfactant- | polymer of |
| | free | formula (II) | free | formula (II) |
| Initial | 71% | 98% | 63% | 107% |
| 12 Week at 40° | 73% | 99% | 62% | 110% |
| C./75% relative | | | | |
| humidity | | | | |

Table 5 shows the variability of individual doses, combining beginning and end of use doses (% Relative Standard Deviation, n=20).

TABLE 5

| | Compound B Component | | Compound A Component | |
|---|---|---|---|---|
| | | 0.1% w/w of | | 0.1% w/w of |
| | Surfactant- | polymer of | Surfactant- | polymer of |
| | free | formula (II) | free | formula (II) |
| Initial | 11.8% | 5.8% | 11.7% | 2.6% |
| 12 Week at 40° | 10.3% | 6.1% | 9.6% | 2.1% |
| C./75% relative | | | | |
| humidity | | | | |

Fine Particle Fraction—Compound A in Combination with a Compound B, MDI, HFA 134a, 25 μg/act, 60 Actuations

Table 6 shows the Fine Particle Fraction expressed as a percentage of the total dose target, for Compound B, 10 μg and for the Compound A, 25 μg.

TABLE 6

| | Compound B Component | | Compound A Component | |
|---|---|---|---|---|
| | | 0.1% w/w of | | 0.1% w/w of |
| | Surfactant- | polymer of | Surfactant- | polymer of |
| | free | formula (II) | free | formula (II) |
| Initial | 20% | 25% | 18% | 32% |
| 12 Week at 40° | 21% | 26% | 18% | 35% |
| C./75% relative | | | | |
| humidity | | | | |

Additional formulations of varying concentrations of biocompatible polymer of the present invention were prepared by similar methods.

### Example 3

Compound A in Combination with Compound C, MDI, 25/12.5 μg/act, 60 Actuations

The cold-filling equipment, which consists of a stainless steel batching vessel with an air-driven mixer and filling valve, was assembled. The propellant was chilled to about −60° C. The batching vessel was chilled to at least −30° C. and about half of the total chilled propellant was added. The propellant was allowed to reach at least −50° C. With the mixer running, 1.8037 g of the biocompatible polymer comprising compounds of formula (II) was added followed by addition of 0.5944 g of 6α,9α-difluoro-17α-[(2-furanylcarbonyl)oxy]-11β-hydroxy-16α-methyl-3-oxo-androsta-1,4-diene-17β-carbothioic acid S-fluoromethyl ester powder and 0.3786 g of 3-(4-{[6-({(2R)-2-hydroxy-2-[4-hydroxy-3-(hydroxymethyl)-phenyl]ethyl}amino)hexyl]oxy}butyl)benzenesulfonamide powder. The remaining cold propellant was then added up to the total weight, 1797 g of HFA 134a, and the containers rinsed to ensure all the powders were added. The suspension was mixed at about 3000 rpm for about 15 minutes. Before filling the MDI units, the formulation temperature was confirmed to be about −60° C. The filling valve was adjusted to deliver the appropriate fill weight. Fluoropolymer coated aluminum canisters were filled to the specific fill weight target, 7.3 g of HFA 134a. A Valois DF60 Mark 66 valve was immediately placed on the canister and crimped. Each unit was formulated to deliver a total of about 100 actuations. The formulations were then allowed to warm to room temperature and spray tested two times to waste, in order to ensure that the unit was working correctly.

Comparative surfactant-free formulations were prepared as generally described above with the exception that no biocompatible polymer of formula (II) was added to the formulation.

Dose Uniformity—Compound A in Combination with Compound C, MDI, HFA 134a, 25/12.5 μg/act, 60 actuations

Table 7 shows the overall mean dose of medicament delivered through the actuator, combining beginning and end of use doses.

The target output of Compound C is 11.3 μg (assuming 10% actuator deposition). The target output of Compound A is 22.5 μg (assuming 10% actuator deposition).

TABLE 7

| | Compound C Component | | Compound A Component | |
|---|---|---|---|---|
| | | 0.1% w/w | | 0.1% w/w |
| | Surfactant- | polymer of | Surfactant- | polymer of |
| | free | formula (II) | free | formula (II) |
| Initial | 80% | 96% | 75% | 101% |
| 12 Week at 40° | 90% | 112% | 76% | 107% |
| C./75 relative | | | | |
| humidity | | | | |

Table 8 shows the variability of individual doses, combining beginning and end of use dose (% Relative Standard Deviation, n=20).

TABLE 8

| | Compound C Component | | Compound A Component | |
|---|---|---|---|---|
| | | 0.1% w/w | | 0.1% w/w |
| | Surfactant- | polymer of | Surfactant- | polymer of |
| | free | formula (II) | free | formula (II) |
| Initial | 6.9% | 3.9% | 7.2% | 3.6% |
| 12 Week at 40° | 10.0% | 4.3% | 9.9% | 3.6% |
| C./75 relative | | | | |
| humidity | | | | |

Fine Particle Fraction—Compound A in Combination with Compound C, MDI, HFA 134a, 25/12.5 μg/act, 60 actuations

Table 9 shows the Fine Particle Fraction (FPF) expressed as a percentage of the total dose target, for Compound C, 12.5 μg and for Compound A, 25 μg.

TABLE 9

| | Compound C Component | | Compound A Component | |
|---|---|---|---|---|
| | | 0.1% w/w | | 0.1% w/w |
| | Surfactant- | polymer of | Surfactant- | polymer of |
| | free | formula (II) | free | formula (II) |
| Initial | 14% | 28% | 11% | 21% |
| 12 Week 40° | 21% | 25% | 16% | 20% |
| C./75 relative | | | | |
| humidity | | | | |

### Example 4

Compound A in Combination with Compound D, MDI 100/100 μg/act, 60 Actuations

The cold-filling equipment, which consists of a stainless steel batching vessel with an air-driven mixer and filling valve, was assembled. The propellant was chilled to about −60° C. The batching vessel was chilled to at least −30° C. and about half of the total chilled propellant was added. The propellant was allowed to reach at least −50° C. With the mixer running, 4.3119 g of the biocompatible polymer comprising compounds of formula (II) was added followed by addition of 5.6562 g of 6α,9α-difluoro-17α-[(2-furanylcarbonyl)oxy]-11β-hydroxy-16α-methyl-3-oxo-androsta-1,4-diene-17β-carbothioic acid S-fluoromethyl ester powder and 9.0090 g of 4-{(1R)-2-[(6-{2-[(2,6-dichlorobenzyl) oxy] ethoxy} hexyl) amino]-1-hydroxyethyl}-2-(hydroxymethyl) phenol powder. The remaining cold propellant was then added up to the total weight, 4294 g of HFA 134a, and the containers rinsed to ensure all the powders were added. The suspension was mixed at about 3000 rpm for about 15 minutes. Before filling the MDI units, the formulation temperature was confirmed to be about −60° C. The filling valve was adjusted to deliver the appropriate fill weight. Fluoropolymer coated aluminum canisters were filled to the specific fill weight target, 7.3 g of HFA 134a. A Bespak BK357930MT valve was immediately placed on the canister and crimped. Each unit was formulated to deliver a total of about 100 actuations. The formulations were then allowed to warm to room temperature and spray tested two times to waste, in order to ensure that the unit was working correctly.

Dose Uniformity—Compound A in Combination with Compound D, HFA-134a MDI 100/100 μg/act, 60 Actuations

Table 10 shows the overall mean dose delivered through the actuator, combining beginning and end of use.

The target output of compound D is 90 μg (assuming 10% actuator deposition)

The target output of compound A is 90 μg (assuming 10% actuator deposition)

TABLE 10

| | Compound D | Compound A |
|---|---|---|
| | Component | Component |
| | 0.1% w/w of polymer | 0.1% w/w of polymer |
| | of formula (II) | of formula (II) |
| Initial | 101% | 106% |
| 12 Week at 40° C./75% | 104% | 111% |
| relative humidity | | |

Table 11 shows the variability of individual doses, combined beginning and end of use (% Relative Standard Deviation, n=20)

TABLE 11

| | Compound D | Compound A |
|---|---|---|
| | Component | Component |
| | 0.1% w/w of polymer | 0.1% w/w of polymer |
| | of formula (II) | of formula (II) |
| Initial | 2.5% | 2.6% |
| 12 Week at 40° C./75% | 3.1% | 2.3% |
| relative humidity | | |

Fine Particle Fraction—Compound A in Combination with Compound D, HFA-134a, MDI 100/100 μg/act, 60 Actuations

Table 12 shows the Fine Particle Fraction (FPF) expressed as a percentage of the total dose target, for Compound D, 100 μg, and for Compound A, 100 μg

TABLE 12

| | Compound D | Compound A |
|---|---|---|
| | Component | Component |
| | 0.1% w/w of polymer | 0.1% w/w of polymer |
| | of formula (II) | of formula (II) |
| Initial | 32% | 21% |
| 12 Week at 40° C./75% | 27% | 19% |
| relative humidity | | |

### Example 5

Compound A in Combination with Compound E, MDI 100/50 μg/act, 60 Actuations

The cold-filling equipment, which consists of a stainless steel batching vessel with an air-driven mixer and filling valve, was assembled. The propellant was chilled to about −60° C. The batching vessel was chilled to at least −30° C. and about half of the total chilled propellant was added. The propellant was allowed to reach at least −50° C. With the mixer running, 3.7025 g of the biocompatible polymer comprising compounds of formula (II) was added followed by addition of 2.4286 g of 6α,9α-difluoro-17α-[(2-furanylcarbonyl)oxy]-11β-hydroxy-16α-methyl-3-oxo-androsta-1,4-diene-17β-carbothioic acid S-fluoromethyl ester powder and 6.9699 g of N-{2-[4-(3-phenyl-4-methoxyphenyl)aminophenyl]ethyl}-2-hydroxy-2-(8-hydroxy-2(1H)-quinolinon-5-yl)ethylamine powder. The remaining cold propellant was then added up to the total weight, 3691 g of HFA 134a, and the containers rinsed to ensure all the powders were added. The suspension was mixed at about 3000 rpm for about 15 minutes. Before filling the MDI units, the formulation temperature was confirmed to be about −60° C. The filling valve was adjusted to deliver the appropriate fill weight. Fluoropolymer coated aluminum canisters were filled to the specific fill weight target, 7.3 g of HFA 134a. A Bespak BK357930MT valve was immediately placed on the canister and crimped. Each unit was formulated to deliver a total of about 100 actuations. The formulations were then allowed to warm to room temperature and spray tested two times to waste, in order to ensure that the unit was working correctly.

Dose Uniformity—Compound E in Combination with Compound A, MDI 100/50 μ/act, 60 Actuations

Table 13 shows the overall mean dose delivered through the actuator, combined beginning and end of use

The target output of Compound E is 90 μg (assuming 10% actuator deposition)

The target output of Compound A is 45 μg (assuming 10% actuator deposition)

TABLE 13

| | Compound E | Compound A |
|---|---|---|
| | Component | Component |
| | 0.1% w/w of polymer | 0.1% w/w of polymer |
| | of formula (II) | of formula (II) |
| Initial | 104% | 104% |
| 12 Week at 40° C./75% | 106% | 109% |
| relative humidity | | |

Table 14 shows the variability of individual doses, combined beginning and end of use (% Relative Standard Deviation, n=20)

TABLE 14

| | Compound E | Compound A |
|---|---|---|
| | Component | Component |
| | 0.1% w/w of polymer | 0.1% w/w of polymer |
| | of formula (II) | of formula (II) |
| Initial | 2.6% | 2.7% |
| 12 Week at 40° C./75% | 1.8% | 1.6% |
| relative humidity | | |

Fine Particle Mass—Compound A in Combination with Compound E, MDI 100/50 μg/act, 60 Actuations

Table 15 shows the Fine Particle Fraction (FPF) expressed as percentage of the total dose target of Compound E, 100 μg and for Compound A, 50 μg

TABLE 15

| | Compound E | Compound A |
|---|---|---|
| | Component | Component |
| | 0.1% w/w of polymer | 0.1% w/w of polymer |
| | of formula (II) | of formula (II) |
| Initial | 26% | 23% |
| 12 Week at 40° C./75% | 28% | 22% |
| relative humidity | | |

Additional formulations of varying concentrations of biocompatible polymer of the present invention were prepared by similar methods.

We consider that the data and the figures show an increase in dose and FPF delivered through the valve as the concentration of the biocompatible polymer in the formulation is increased up to a concentration of at least 0.1% w/w relative to propellant.

The data also appear to show that the addition of a biocompatible polymer of the present invention to a pharmaceutical aerosol formulation leads to reduced variability of individual doses.

1. A pharmaceutical aerosol formulation comprising:
i) a therapeutically effective amount of particulate medicament of formula (I) 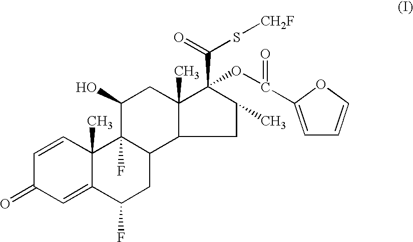
or a solvate thereof;
(ii) a propellant selected from the group consisting of 1,1,1,2-tetrafluoroethane or 1,1,1,2,3,3,3-heptafluoro-n-propane or mixtures thereof; and
(iii) a biocompatible polymer comprising one or more compounds of formula (II) 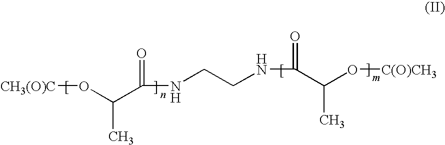
wherein n and m independently represent an integer of at least one and the independent average value of n and m in the biocompatible polymer is between 6 and 25; and each unit of formula 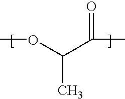
is independently in the D or L configuration.

2. A pharmaceutical aerosol formulation as claimed in claim 1 wherein the independent average value of n and m in the biocompatible polymer is between 7 and 11.

3. A pharmaceutical aerosol formulation as claimed in claim 1 in which the particulate medicament of formula (I) is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

4. A pharmaceutical aerosol formulation as claimed in claim 1 characterised in that the compound of formula (I) is in unsolvated form.

5. A pharmaceutical aerosol formulation as claimed in claim 4 wherein the compound of formula (I) is in the form of Form 1 polymorph.

6. A pharmaceutical aerosol formulation as claimed in claim 1 in which the propellant is 1,1,1,2-tetrafluoroethane.

7. A pharmaceutical aerosol formulation as claimed in claim 1 in which the propellant is 1,1,1,2,3,3,3-heptafluoro-n-propane.

8. A pharmaceutical aerosol formulation as claimed in claim 1 further comprising one or more other therapeutically active agents.

9. A pharmaceutical aerosol formulation as claimed in claim 8 in which said another therapeutically active agent is a β₂-adrenoreceptor agonist.

10. A pharmaceutical aerosol formulation as claimed in 9 wherein the β₂-adrenoreceptor agonist is selected from:
salmeterol; (R)-salmeterol; salbutamol; (R)-salbutamol; formoterol; (R,R)-formoterol; fenoterol; carmoterol; etanterol; naminterol; clenbuterol; pirbuterol; flerobuterol; reproterol; bambuterol; terbutaline; salmefamol; indacaterol;
3-(4-{[6-({(2R)-2-hydroxy-2-[4-hydroxy-3-(hydroxymethyl)phenyl]ethyl} amino) hexyl] oxy} butyl) benzenesulfonamide;
3-(3-{[7-({(2R)-2-hydroxy-2-[4-hydroxy-3-hydroxymethyl) phenyl] ethyl} -amino) heptyl] oxy} propyl) benzenesulfonamide;
4-{(1R)-2-[(6-{2-[(2,6-dichlorobenzyl) oxy] ethoxy} hexyl) amino]-1-hydroxyethyl}-2-(hydroxymethyl) phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-{2-[4-(3-phenyl-4-methoxyphenyl)aminophenyl]ethyl}-2-hydroxy-2-(8-hydroxy-2(1H)-quinolinon-5-yl)ethylamine;
5-[(R)-2-(2-{4-[4-(2-amino-2-methyl-propoxy)-phenylamino]-phenyl}-ethylamino)-1-hydroxyethyl]-8-hydroxy-1H-quinolin-2-one; and
pharmaceutically acceptable salts thereof.

11. A pharmaceutical aerosol formulation as claimed in claim 10 wherein the β₂-adrenoreceptor agonist is selected from salmeterol and (R)-salmeterol.

12. A pharmaceutical aerosol formulation as claimed in claim 9 wherein the β₂-adrenoreceptor agonist is in the form of a salt formed with a pharmaceutically acceptable acid selected from sulphuric, hydrochloric, fumaric, hydroxynaphthoic, cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

13. A pharmaceutical aerosol formulation as claimed in claim 12 wherein the β₂-adrenoreceptor agonist is salmeterol xinafoate (1-hydroxy-2-naphthalenecarboxylate).

14. A pharmaceutical aerosol formulation as claimed in claim 12 wherein the β₂-adrenoreceptor agonist is salbutamol sulphate.

15. A pharmaceutical aerosol formulation as claimed in claim 12 wherein the β₂-adrenoreceptor agonist is formoterol fumarate.

16. A process for the preparation of an aerosol formulation as claimed in claim 1 which comprises dispersal of the medicament of formula (I) and the biocompatible polymer comprising one or more compounds of formula (II) in the propellant in an appropriate container.

17. A pharmaceutical aerosol formulation as claimed in claim 1 for use in veterinary or human medicine.

18.-20. (canceled)

21. A method of treatment or prophylaxis of a respiratory disorder which comprises administering to a human or animal subject a pharmaceutical aerosol formulation as claimed in claim.

22. A metered dose inhaler containing therein a pharmaceutical aerosol formulation according to claim 1.

23. A metered dose inhaler as claimed in claim 22 wherein the whole of the internal metallic surface of the can is coated with a polymer blend of polytetrafluoroethylene and polyethersulfone.

24.-26. (canceled)
